(19) United States
(12) Patent Application Publication
(10) Pub. No.: US 2021/0369983 A1
(43) Pub. Date: Dec. 2, 2021

(54) A NEEDLE DELIVERY DEVICE

(71) Applicant: NDM TECHNOLOGIES LIMITED, Leicester (GB)

(72) Inventor: Dewan Fazlul Hoque CHOWDHURY, Leicester (GB)

(73) Assignee: NDM TECHNOLOGIES LIMITED, Leicester (GB)

(21) Appl. No.: 16/322,480

(22) PCT Filed: Jul. 28, 2017
(86) PCT No.: PCT/GB2017/052211

§ 371 Date: Jan. 31, 2019

(30) Foreign Application Priority Data
Aug. 1, 2016 (GB) 1613215.1

(51) Int. Cl. A61M 5/46; A61M 5/158

(52) U.S. Cl. CPC A61M 5/46; A61M 2205/10; A61M 2005/1585; A61M 5/158

(57) ABSTRACT

A needle delivery device (10; 100) comprises a device body (12) that has an opening (14) formed on a delivery surface (16) of the device body (12). The needle delivery device (10; 100) also includes a drive mechanism (28) which is configured to, in use, drive a needle (20; 120), at least a portion (22) of which is flexible, along a needle path (30) towards the opening (14). The needle path (30) changes direction as it reaches the opening (14). Furthermore, the needle delivery device (10; 100) includes a rigid support member (32) that is fixedly secured to the device body (12) at the opening (14). The rigid support member (32) is configured to guide the needle (20) as it leaves the opening (14) so as to prevent buckling of the needle (20; 120). The device also comprises a vial (40) for containing a pharmaceutical composition, the vial being in fluid communication with one end of the needle, wherein the vial is moveable in tandem with the needle as the needle is driven along the needle path.

This invention relates to a needle delivery device.

It is known to deliver drugs through the skin, i.e. transdermally, by using a needle in conjunction with a delivery device.

Flexible needles are generally known in the current state of the art as needles that do not maintain a rigid linear structure when a bending force is applied, such as acupuncture needles. Therefore, by definition flexible needles are high aspect ratio, as low aspect ratio needles are generally very rigid and do not bend unless applied forces are substantially greater than that which would be the norm for their intended use. Flexible needles can be produced to allow a 90 degree bend radius without fracturing or permanently deforming the needle. Such needles may be formed using metal alloys such as nickel titanium, stainless steel metals of very high aspect ratio, other inert metals, polymers such as nylon and polyester and medical grade polymers widely used in the construction of syringes. Flexible needles made from these materials would normally be produced through an extrusion mechanism. A combination of a metal tip and plastic body/conduit may also be used to form a flexible needle, wherein the metal tip provides a sharp mechanically strong leading tip. Flexible needles may be as small as 10's of microns thin, such as hollow fiber optic cables, through to standard 18 gauge at the tip, with equivalent or different diameter flexible conduit connected to the reservoir via a luer slip or luer lock connection, via which the drug may flow from the reservoir.

The word device is used herein to describe a system or structure into which a needle is loaded. The word needle is used here to describe one or more needles and associated housing and conduits where present, whereby the needle may be a single unit or formed from multiple units joined together to provide a rigid portion and a flexible portion. The term vial is used to describe a reservoir for the storage of a drug or other pharmaceutical agent, biological drug, vaccine, or cosmetic agent, intended for administration into a patient who may be a human or animal subject.

According to an aspect of the invention there is provided a needle delivery device comprising:
a device body having an opening formed on a delivery surface of the device body;
a drive mechanism configured to, in use, drive a needle, a portion of which is flexible, along a needle path towards the opening, the needle path changing direction as it reaches the opening;
a rigid support member fixedly secured to the device body at the opening, the rigid support member being configured to guide the needle as it leaves the opening so as to prevent buckling of the needle; and
a vial for containing a pharmaceutical composition, the vial being in fluid communication with one end of the needle, wherein the vial is moveable in tandem with the needle as the needle is driven along the needle path.

Having a needle path which changes direction as it reaches the opening means that the end of a needle being delivered does not have to be in line with the other end of the needle. It also means that the height of the needle delivery device is not dictated by the needle length. As such, the needle delivery device can have a low profile compared to conventional devices.

The term opening defines a region within the device body at which point a needle extends beyond the device so as to contact the surface of the skin when the device is in use.

Such a conventional device will typically inject the needle vertically relative to the skin of a patient such that the device, when being used, stands proud of the skin. When a long needle is to be used, such as an intramuscular injection needle, a conventional device will stand proud of the skin by a height that is at least equal to the length of the needle. This results in a tall, cumbersome device.

Returning to the present invention, the inclusion of a rigid support member that prevents buckling of the needle as it leaves the opening reduces the risk of the needle being damaged or broken as it leaves the device and is being delivered to the skin. This can be particularly important when the needle is long, e.g. an intramuscular injection needle, and so is more likely to buckle. The rigid support member thus helps to ensure that the lumen of the needle remains open and unblocked.

Optionally the needle path is at an angle less than 90° to the delivery surface of the device body before changing direction. Indeed the needle path may be at an angle less than 45° to the delivery surface of the device body before changing direction.

Preferably the needle path is substantially parallel to the delivery surface of the device body before changing direction.

Such arrangements of the needle path provide the desired low profile needle delivery device.

In some embodiments of the invention the needle delivery device further includes a needle, at least a portion of the needle being flexible to permit a change in direction of the needle along the needle path.

The needle may be an intramuscular injection needle. Indeed the needle may be configured to be delivered to a depth of at least 25.4 mm (1″). Such a depth is typically necessary to achieve deep tissue or intramuscular injections.

An intramuscular injection is a technique used to deliver medicine deep into the muscles. It is used for specific types of medication and to achieve a fast rate of absorption than other types of injection, e.g. intradermal injections. Typical injection sites for intramuscular injections are the deltoid muscle of the arm, the vastus lateralis muscle of the thigh and the dorsogluteal muscles of the buttocks. Since the needle for an intramuscular injection must be able to reach the muscle without penetrating the nerves and blood vessels underneath, the needle must typically be at least 25.4 mm (1″) in length, and is normally between 25.4 mm and 38.1 mm (1.5″) to achieve the desired penetration depth. The needle also typically has a gauge of around 22 to 25.

The rigid support member may extend from the opening. As such, the rigid support member is external to the device body and so provides support to the needle as it leaves the device and enters a patient's skin.

Optionally the needle delivery device includes a guide member configured to guide the needle along the needle path.

The inclusion of a guide member helps with travel of the needle along the needle path.

The guide member may be or include a roller and/or pulley. Such an arrangement provides an effective means of guiding the needle along the needle path.

The needle delivery device includes a vial for containing a pharmaceutical composition, the vial being in fluid communication with one end of the needle, wherein the vial is moveable in tandem with the needle as the needle is driven along the needle path.

The vial being in fluid communication with one end of the needle permits effective delivery of the pharmaceutical composition from the vial to the patient. Meanwhile, the vial moving in tandem with the needle maintains that fluid communication as the needle is driven along the needle path.

Preferably the needle delivery device further includes a direction-changing member positioned at the opening and configured to change the direction of the needle path as it reaches the opening.

The direction-changing member may be integrally formed with the rigid support member. This reduces the component count of the needle delivery device.

Preferably the rigid support member is a sleeve. Such an arrangement readily permits the rigid support member to surround the needle as it leaves the opening. The sleeve may be a single component that fully surrounds the needle or instead might be made from two or more components that partially surround the needle, e.g. two half-pipes that surround either side of the needle.

According to another aspect of the invention there is provided a needle delivery device assembly comprising:
a needle delivery device including a needle as described hereinabove; and
a vial containing a pharmaceutical composition, the vial being in fluid communication with the needle.

According to a further aspect of the invention there is provided a method of administering a pharmaceutical composition to a patient using a needle delivery device assembly as described hereinabove.

There now follows a brief description of preferred embodiments of the invention, by way of non-limiting examples, with reference being made to the accompanying drawings in which:

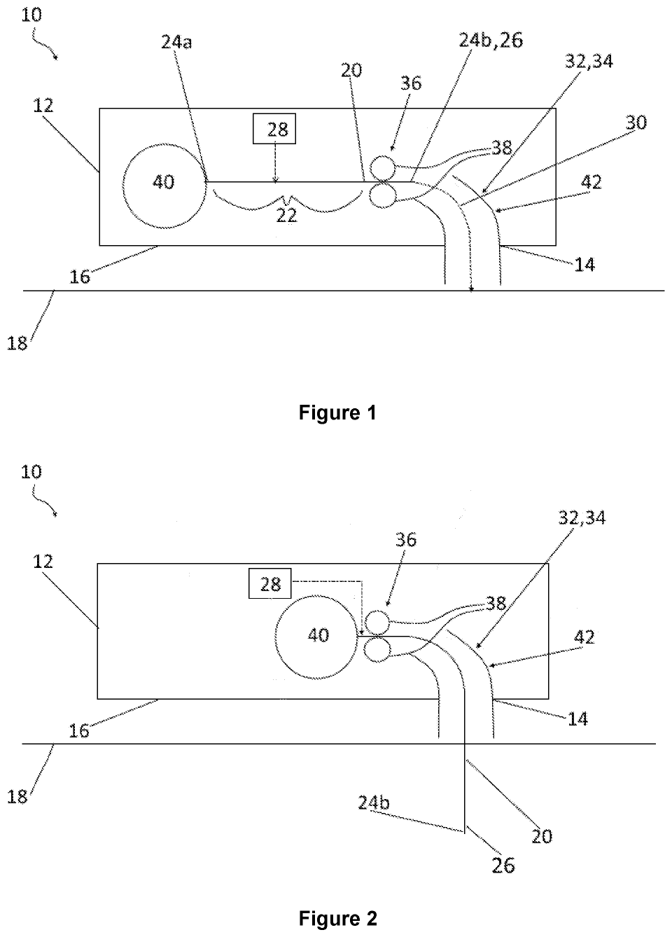

FIG. 1 shows a schematic illustration of a needle delivery device in a first position according to a first embodiment of the invention;

FIG. 2 shows a schematic illustration of the needle delivery device shown in FIG. 1 in a second position;

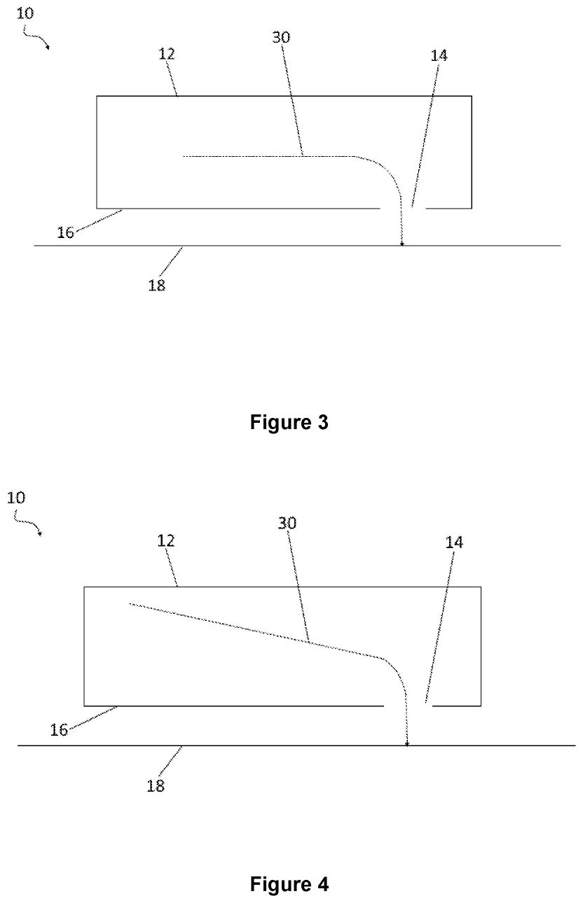

FIG. 3 shows a schematic illustration of the needle pathway of the needle delivery device shown in FIG. 1;

FIG. 4 shows a schematic illustration of an alternative needle pathway; and

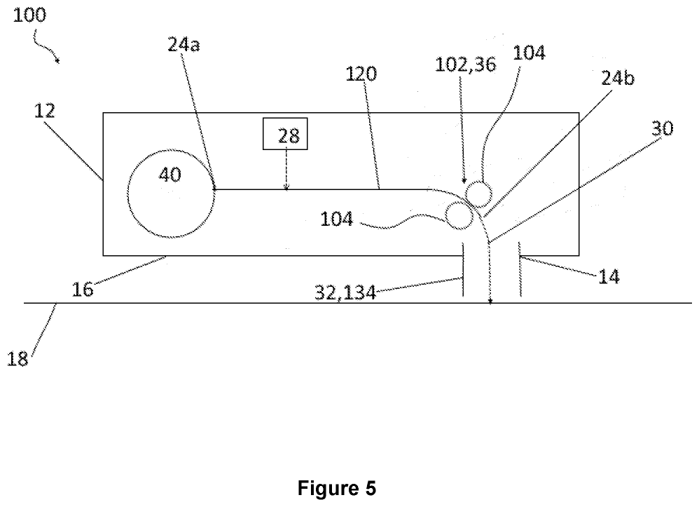

FIG. 5 shows a schematic illustration of a needle delivery device according to a second embodiment of the invention.

A needle delivery device according to a first embodiment of the invention is shown in FIGS. 1 to 3 and is designated generally by reference numeral 10.

The first needle delivery device 10 includes a device body 12 which has an opening 14 formed on a delivery surface 16 of the device body 12. The delivery surface 16 is the surface of the device body 12 which faces the skin 18 of a patient when the first needle delivery device 10 is being used to deliver a pharmaceutical composition.

The first needle delivery device 10 shown also includes a needle 20 which has a flexible portion 22 extending along its length. The flexible portion 22 permits a change in direction of the needle 20, i.e. it permits the needle 20 to bend by at least 45° and preferably by 90°. The needle 20 includes opposing first and second ends 24a, 24b. The first end 24a is configured to be in fluid communication with a vial 40 (described in more detail below) and the second end 24b is injected into the skin 18 of a patient.

The needle 20 is made from a flexible plastic conduit to form the flexible portion 22 and a rigid needle tip 26 at the second end 24b of the needle 20. Instead, the entire needle 20 may be flexible by being formed from a flexible material such as a thin metal.

The delivery surface 16 is shown in this embodiment to be spaced from the skin surface 18. In other embodiments however the two surfaces 18, 16 may be in direct contact to provide a firm mounting of the device on the skin prior to insertion of the needle 20 into the skin 18.

The first needle delivery device 10 further includes a drive mechanism 28 which is configured to drive the needle 20 along a needle path 30 towards the opening 14. The needle path 30 changes direction as it reaches the opening.

In particular, the drive mechanism 28 drives the needle 20 from a first position (as shown in FIG. 1) wherein the needle 20 is inside the device body 12, to a second position (as shown in FIG. 2) wherein the second end 24b of the needle 20 has left the device body 12 via the opening 14. In this regard, it is the second end 24 of the needle 20 that changes direction as it is driven towards the opening 14.

The drive mechanism 28 may be mechanically operated using a series of rollers, pulleys and suitably mounted springs, or it may be electromechanically driven using actuators such as a combination of motors and gears, or linear actuators such as those constructed from shape memory metals.

Furthermore, the first needle delivery device 10 includes a rigid support member 32 which is fixedly secured to the device body 12 and extends from the opening 14. The rigid support member 32 is configured to surround the needle 20 at an appropriate distance up to the opening 14, and/or as it leaves the opening 14 so as to prevent buckling of the needle 20.

In the embodiment shown, the rigid support member 32 is a sleeve 34 which extends partially within the device body 12 and partially out of the device body 12 at the opening 14. The rigid support member 32 may take another form and may only extend either within or out of the device body 12 at the opening 14.

The rigid support member 32 may comprise a low friction material, such as a lubricious coating, to facilitate the passage of the needle 20.

As shown more clearly in FIG. 3, the needle path 30 of this embodiment runs substantially parallel to the delivery surface 16 of the device body 12 before changing direction. As such, the needle delivery device 10 has a low profile since its height, relative to the skin 18 when the device 10 is being used, is not dictated by the length of the needle 20.

The needle path 30 changes direction at approximately 90° such that the second end 24b of the needle 20 is perpendicular to the needle path 30 before it changes direction. In this way, the needle 20 is delivered at 90° to the skin 18, which is preferable for certain types of injection, e.g. intramuscular injection. The needle path 30 may instead change direction at a different angle such that the needle 20 is delivered to the skin 18 at an angle other than 90°.

An alternative needle path 30 is shown in FIG. 4 which is a needle path 30 at approximately 45° to the delivery surface 16 before changing direction to deliver the needle 20 at 90° to the skin 18.

In the embodiment shown, the needle 20 is an intramuscular injection needle and so has the properties required to perform an intramuscular injection. These properties, such as length of the needle and depth of penetration of the needle into the skin, are known in the art. For example, certain types of intramuscular injections require a depth of penetration of around 38 mm (1.5″). The length of the needle 20 will therefore be suitable to deliver such a depth of penetration.

The first needle delivery device 10 further includes a guide member 36 in the form of a pair of rollers 38. The guide member 36 guides the needle 20 along the needle path 30 by the rollers 38 rotating and thus helping to push the needle 20 towards the opening 14.

There may instead only be one roller 38. The guide member 36 may take another form, such as a pulley.

The first needle delivery device 10 also includes a vial 40 for containing a pharmaceutical composition (not shown), e.g. a drug. The vial 40 may be a pre-filled syringe, or a vial with a double septum (one at each end) or some other bespoke reservoir that contains the drug.

The vial 40 is in fluid communication with the first end 24a of the needle 20. This may be a direct fluid communication or an indirect fluid communication via an intermediate member which permits such fluid communication. For example, there may be a flexible conduit connected at one end of the vial 40 via a luer slip or luer lock connection, and at the other end to the needle 20.

The vial 40 is moveable in tandem with the needle 20 as the needle 20 is driven along the needle path 30. The drive mechanism 28 may also be configured to drive the vial 40 along the needle path 30.

In any event, the vial 40 is moved from a first position, as shown in FIG. 1, to a second position, as shown in FIG. 2. Once the needle 20 has penetrated the skin 18, the pharmaceutical composition is delivered to the patient from the vial in a conventional manner, e.g. via a plunger mechanism that forces the contents of the vial or pre-filled syringe through the bore of the needle 20. Where alternative collapsible reservoirs are used, containing a fluid communication connection with a suitably mounted needle, the contents of the reservoir may be dispensed by compressing the reservoir leading it to collapse as the content is expelled.

In the embodiment shown, the rigid support member 32 has an integrally formed direction-changing member in the form of a bend 42 in the sleeve 34. The bend 42 forces the needle 20 to change direction as desired. As such, the bend 42 can be formed so as to dictate the severity of the change in direction of the needle path 30 depending on the requirements of the needle delivery device 10.

A needle delivery device 100 according to a second embodiment of the invention is shown in FIG. 5 and is designated generally by reference numeral 100.

The second needle delivery device 100 shares features with the first needle delivery device 10 and identical features are indicated by the same reference numeral.

The second needle delivery device 100 differs from the first needle delivery device 10 in that it includes a direction-changing member 102 that is separate from the rigid support member 32.

The direction-changing member 102 is in the form of a pair of rollers 104 which are positioned to force the needle 20 to change direction when they pass therethrough. In the embodiment shown, the rollers 104 also act as the guide member 36. However in other embodiments of the invention the needle delivery device 100 may include a separate guide member 36 in addition to the rollers 104.

Since the direction-changing member 102 is separate from the rigid support member 32, the rigid support member 32 no longer requires a bend and so it takes the form of a sleeve 134 with straight edges.

The separate direction-changing member 102 may take any form which forces the needle path 30 to change direction as it approaches the opening 14.

The second needle delivery device 100 also differs from the first needle delivery device 10 in that the needle 120 is flexible along its entire length. The needle 120 may be formed from using metal alloys such as nickel titanium, stainless steel metals of very high aspect ratio, other inert metals, polymers such as nylon and polyester and medical grade polymers widely used in the construction of syringes. The needle 120 is formed through an extrusion process.

The needle 120 may instead have a flexible portion (e.g. a plastic body/conduit) and a rigid metal tip, such as that described hereinabove with reference to the first needle delivery device 10.

1. A needle delivery device comprising:
a device body having an opening formed on a delivery surface of the device body;
a drive mechanism configured to, in use, drive a needle, at least a portion of which is flexible, along a needle path towards the opening, the needle path changing direction as it reaches the opening;
a rigid support member fixedly secured to the device body at the opening, the rigid support member being configured to guide the needle as it leaves the opening so as to prevent buckling of the needle; and
a vial for containing a pharmaceutical composition, the vial being in fluid communication with one end of the needle, wherein the vial is moveable in tandem with the needle as the needle is driven along the needle path.

2. The needle delivery device according to claim 1 wherein the needle path is at an angle less than 90° to the delivery surface of the device body before changing direction.

3. The needle delivery device according to claim 1 wherein the needle path is at an angle less than 45° to the delivery surface of the device body before changing direction.

4. The needle delivery device according to claim 1 wherein the needle path is substantially parallel to the delivery surface of the device body before changing direction.

5. The needle delivery device according to claim 1 further including a needle, at least a portion of the needle being flexible to permit a change in direction of the needle along the needle path.

6. The needle delivery device according to claim 5 wherein the needle is an intramuscular injection needle.

7. The needle delivery device according to claim 5 wherein the needle is configured to be delivered to a depth of at least 25.4 mm (1″).

8. The needle delivery device according to claim 1 wherein the rigid support member extends from the opening.

9. The needle delivery device according to claim 1 further including a guide member configured to guide the needle along the needle path.

10. The needle delivery device according to claim 9 wherein the guide member is or includes at least one of a roller or a pulley.

11. The needle delivery device according to claim 1 further including a direction-changing member positioned at the opening and configured to change the direction of the needle path as it reaches the opening.

12. The needle delivery device according to claim 11 wherein the direction-changing member is integrally formed with the rigid support member.

13. The needle delivery device according to claim 1 wherein the rigid support member is a sleeve.

14. A needle delivery device assembly comprising:
a needle delivery device comprising:
a device body having an opening formed on a delivery surface of the device body;
a drive mechanism configured to, in use, drive a needle, at least a portion of which is flexible, along a needle path towards the opening, the needle path changing direction as it reaches the opening;
a rigid support member fixedly secured to the device body at the opening, the rigid support member being configured to guide the needle as it leaves the opening so as to prevent buckling of the needle;
a vial for containing a pharmaceutical composition, the vial being in fluid communication with one end of the needle, wherein the vial is moveable in tandem with the needle as the needle is driven along the needle path; and
a needle, at least a portion of the needle being flexible to permit a change in direction of the needle along the needle path; and
a vial containing a pharmaceutical composition, the vial being in fluid communication with the needle.

15. A method of administering a pharmaceutical composition to a patient using a needle delivery device assembly according to claim 14.

16-17. (canceled)
